(19) United States
(12) Patent Application Publication
(10) Pub. No.: US 2011/0052537 A1
(43) Pub. Date: Mar. 3, 2011

(54) NITRO-BASED EXPLOSIVE REMEDIATION

(76) Inventors: A. Morrie Craig, Corvallis, OR (US); Karen Walker, Philomath, OR (US); Sudeep Perumbakkam, Corvallis, OR (US); Jennifer Duringer, Brownsville, OR (US); Marthah Delorme, Corvallis, OR (US)

(21) Appl. No.: 12/673,850

(22) PCT Filed: Aug. 15, 2008
(86) PCT No.: PCT/US08/73396

§ 371 Date: Feb. 17, 2010

Related U.S. Application Data
(60) Provisional application No. 60/965,158, filed on Aug. 17, 2007.

(51) Int. Cl. A61K 35/74; C12N 1/20; C12Q 1/68; A61P 43/00; A62D 3/02

(52) U.S. Cl. 424/93.3; 435/252.1; 435/6; 435/262.5

(57) ABSTRACT

Embodiments of techniques for remediating soil contaminated with compounds, particularly nitro-based explosives, are disclosed. Plants capable of taking up the compounds are grown within contaminated soil for a period of time. The plants are exposed to anaerobic microbes in the rumen of a ruminant animal. The ruminal anaerobic microbes degrade the remediable compounds and render them substantially nontoxic to the animal. In some embodiments, the remediable compounds are nitroaromatic compounds, and are degraded by nitroreductases. In other embodiments, microbes are transferred from a consortium enriched in microbes capable of degrading the remediable compounds to a ruminant animal that lacks microbes capable of degrading the remediable compounds. In other embodiments, methods for isolating and identifying ruminal anaerobic microbes capable of degrading remediable compounds are disclosed.

### CROSS REFERENCE TO RELATED APPLICATION

This application claims the benefit of the earlier filing date of U.S. provisional application No. 60/965,158, entitled “Munition Bioremediation by Ruminal Microbes and Cool-Season Grasses,” naming Professor A. Morrie Craig as the inventor, and filed on Aug. 17, 2007, which is incorporated in its entirety herein by reference.

### ACKNOWLEDGMENT OF GOVERNMENT SUPPORT

This invention was made with government support under Research Accounting Index No. R0293A awarded by the United States Department of Agriculture, grant No. 58-1256-6-076 awarded by the United States Department of Agriculture/Agricultural Research Service. Also, Hatch Act grant No. ATX521 ACRA awarded by Agricultural Experiment Service. The United States government has rights in the invention.

### FIELD

The disclosure relates generally to remediation of nitro-based explosives, such as remediation of compounds found in military ordnance.

### BACKGROUND

Many explosives currently used by the military and industry are nitroaromatic compounds. A nitroaromatic compound is an aromatic compound having at least one nitro (—NO₂) group attached directly to the aromatic moiety. Generally, aromatic compounds are unsaturated, cyclic hydrocarbons having alternate single and double bonds. Benzene, a 6-carbon ring containing three double bonds, is a typical aromatic compound. One example of a nitroaromatic compound is trinitrotoluene (TNT), the structure of which is provided below.

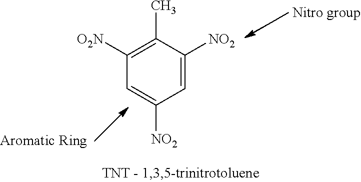

Certain heterocyclic compounds, such as the triazines, also are considered to be aromatic. Triazines are characterized by a 6-membered heterocylic ring having alternating carbon and nitrogen atoms in the ring. Each nitrogen atom has a non-bonding, or lone pair, of electrons, thus providing the triazine molecule with aromatic characteristics. Trinitrotriazine also is a common explosive, and the chemical structure for this compound is provided below.

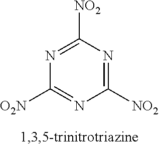

It has been estimated that over 700,000 cubic yards of soil and 10 billion gallons of groundwater require treatment for removal of contaminants at tremendous costs to the DoD. TNT and Royal Demolition Explosive (RDX) are the primary contaminants at these sites, along with dinitrotoluenes (DNT) and the other nitro-substituted explosives (e.g., HMX, tetryl, C4). TNT and RDX, their metabolites, and related compounds are toxic. Nitroaromatic compounds have the ability to rapidly penetrate the skin. Exposure can cause, for example, local irritation, anemia, liver damage, and bladder tumors. Anemia, abnormal liver function, spleen enlargement, harmful effects to the immune system, and adverse effects on male fertility have been identified in animals exposed to TNT.

Nitroaromatic compounds are widespread contaminants on greater than 16,000 Department of Defense (DoD) facilities. Military ranges have substantial contamination levels of nitro-substituted explosives. The cleanup of unexploded ordnance on military ranges has the potential to be the largest environmental cleanup program ever to be implemented in the United States.

Current approaches used for site remediation typically involve excavation of contaminated soils, followed by incineration or composting, and are estimated to cost anywhere from $14 billion to several times that amount to the U.S. taxpayer (Loeb 2002). Bioremediation technologies that have already been employed include windrow composting ($766/ton, Haselhorst 2007), mixing a select consortium of bacteria with contaminated soil ($1,000/cubic yard, U.S. Army Environmental Command 2007), chemical-biological treatment ($1,578/cubic yard, U.S. Army Environmental Command 2007), chemical alteration of soils to enhance microbial activity ($476/cubic yard, U.S. Army Environmental Command 2007), mixing contaminated soil with white rot fungus ($804/cubic yard, U.S. Army Environmental Command 2007) and ex situ anaerobic bioremediation with soil microbes ($112/cubic yard, U.S. Environmental Protection Agency 1995). In addition to the expense, each of these strategies involves excavating, or otherwise disturbing the soil, thus potentially exposing workers to the contaminants in the soil. Accordingly, there remains a need in the art to develop economical and environmentally sound bioremediation technologies.

Certain of the techniques described above have been patented. For example, Crawford et al. isolated and identified three individual strains of anaerobic microoganisms with “an ability to degrade nitroaromatic and nitramine compounds under anaerobic conditions. The strains, identified as LJP-1, SBF-1, and KMR-1, appear to be of Clostridium bifermentans.” (U.S. Pat. No. 5,455,173).
The isolated strains, either individually or as mixtures thereof, can be used in methods for degrading, under anaerobic conditions (i.e., redox potential←200 MV), a contaminant nitroaromatic and/or nitramine compound in water or soil (as an aqueous slurry, i.e., “fluid medium”).

In related U.S. Pat. No. 6,348,639, Crawford et al. also disclose methods for biodegradation of nitroaromatic compounds in water and soils:
[S]oil or water contaminated with one or more nitroaromatic compounds is subjected to a two-stage bioremediation process employing different microorganisms during each stage. The stages comprise an initial fermentation stage followed by an anaerobic stage. Most of the actual biodegradation of the contaminant nitroaromatics takes place in the anaerobic stage. At the end of the anaerobic stage, the contaminant nitroaromatics have been biodegraded to nontoxic end products.

Previously, Fleischmann et at investigated the degradation of TNT by bovine ruminal fluid. [Biochem. and Biophys. Res. Comm., 314 (2004) 957-963.] Whole rumen fluid contents were spiked with TNT and incubated for a 24-hour time period. The study found that:
Within 1 h, TNT was not detectable and reduction products of TNT including 2-hydroxyl-amino-4,6-dinitrotoluene, 4-hydroxylamino-2,6-dinitrotoluene, and 4-amino-2,6-dinitrotoluene were present with smaller amounts of diamino-nitrotoluenes. Within 2 h, only the diamino and dihydroxamino-nitrotoluene products remained. After 4 h, 2,4-diamino-6-nitrotoluene and 2,4-dihydroxyamino-6-nitrotoluene were the only known molecular species left. At 24 h known UV absorbing metabolites were no longer detected.

However, each of the above-described methods is an in vitro process, requiring excavation of the soil and exposing workers to the hazards associated with the contaminants. Thus, a need remains for a bioremediation technology that can be performed on-site without soil excavation or exposing workers to hazardous contaminants.

### SUMMARY

Disclosed herein are embodiments of techniques for remediating remediable compounds, such as environmental contaminants, particularly nitro-based explosives. In some embodiments, areas of land contaminated with remediable compounds are identified. Plants capable of taking up, or absorbing, the remediable compounds are grown within the soil for a period of time. The remediable compounds within the plants are rendered substantially nontoxic to mammals by exposing the plants to anaerobic microbes capable of degrading the remediable compounds. The remediable compounds can include nitroaliphatic compounds, nitroaryl compounds, nitro-heteroaryl compounds, or combinations thereof. In particular embodiments, the plants are grasses, such as cool-season grasses and particularly cool-season grasses used in the dairy industry.

In particular embodiments, the plants are exposed to anaerobic microbes in the rumen of a ruminant animal. The ruminal anaerobic microbes degrade the remediable compounds and render them substantially nontoxic. In some embodiments, the ruminants are sheep. In certain embodiments, the remediable compounds are nitroaromatic compounds, and the nitroaromatic compounds are degraded by nitroreductase enzymes produced by the anaerobic microbes.

In other embodiments, anaerobic microbes are transferred to a ruminant animal that lacks endogenous microbes capable of degrading the remediable compounds. In some embodiments, a consortium of anaerobic microbes in an animal's rumen is enriched for microbes capable of degrading a remediable compound or compounds, such as by exposing the ruminant to the remediable compound or compounds in its diet. In particular embodiments, microbes from the enriched consortium are transferred to a ruminant animal that lacks endogenous microbes capable of degrading the remediable compound or compounds.

In some embodiments, methods for isolating and identifying anaerobic microbes capable of degrading remediable compounds are disclosed. A consortium of anaerobic microbes is obtained, such as from a rumen, and enriched for microbes capable of degrading a remediable compound or compounds, such as nitroaliphatic, nitroaryl, and nitro-heteroaryl compounds or combinations thereof. Individual anaerobic microbes capable of degrading the remediable compound or compounds are isolated and identified. In certain embodiments, the identified anaerobic microbes can be transferred to a ruminant animal lacking in endogenous microbes capable of degrading the remediable compound or compounds.

The foregoing and other objects, features, and advantages of the invention will become more apparent from the following detailed description, which proceeds with reference to the accompanying figures.

### BRIEF DESCRIPTION OF THE DRAWINGS

FIG. 1 is a series of HPLC chromatograms showing degradation of TNT by bovine ruminal fluid over time.

FIG. 2 is a series of autoradiographs illustrating uptake of ¹⁴C-labeled TNT by grasses.

FIG. 3 is a graph of RDX concentration versus time.

### DETAILED DESCRIPTION

### I. Abbreviations

The disclosed embodiments are best understood with reference to the following abbreviations:

ADNT—aminodinitrotoluene

C4—RDX explosive, which has been plasticized to be adhesive and malleable

DANT—diaminonitrotoluene

DNT—dinitrotoluene

GTN—glyceryl trinitrate, commonly known as nitroglycerin

HADNT—hydroxyaminodinitrotoluene

HMX—high molecular weight RDX: 1,3,5,7-tetranitro-1,3,5,7-tetrazocane, also known as octogen, the structure for which is provided below.

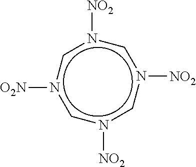

NADH—nicotinamide adenine dinucleotide

NADPH—nicotinamide adenosine dinucleotide phosphate

RDX—Royal Demolition Explosive: 1,3,5-trinitro-1,3,5-triazine; other names for RDX include cyclotrimethylenetrinitramine, cyclonite, hexogen, and T4

Tetryl—2,4,6-trinitrophenyl-N-methylnitramine

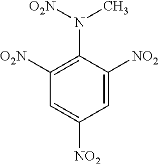

TNT—trinitrotoluene

### II. Bioremediation Techniques

The present disclosure provides embodiments of economical, environmentally sound technologies for remediating remediable compounds, such as environmental contaminants, particularly nitro-based explosives. For certain embodiments, remediation comprises bioremediation, such as by using ruminal microbes. Again in certain embodiments, bioremediation involves degradation of remediable compounds by ruminal metabolization of ingested foods, including plants, such as grass or grasses, and even more typically cool-season grasses. An area may be contaminated with explosives by any of various mechanisms, including production. Moreover, when ordnance explodes in a given area, such as a bombing range or industrial site, a portion of the explosive compound or compounds typically remains unreacted, or nitro-based side products are produced, that contaminates the soil in the area. The soil requires treatment to remove the contaminants. Embodiments of the disclosed methods eliminate the need to excavate, transport, and then process large volumes of contaminated soil, with the potential to reduce clean-up costs by up to 90% and save billions of dollars.

A. Nitro-Based Explosives

Explosives are used by the military and in industry. An explosive is a chemical compound, often containing nitrogen, that detonates (i.e., undergoes extremely rapid, self-propagating decomposition accompanied by a high pressure-temperature wave that moves at thousands of meters per second) as a result of shock or heat. For example, many munitions contain nitro-based explosives. Munitions are materials used in war, primarily weapons and ammunition.

Munitions and other explosives commonly contain nitro-substituted compounds. These compounds may be nitroaliphatic, nitroaryl, or nitro-heteroaryl compounds. For example, glyceryl trinitrate (GTN, nitroglycerin) is a nitroaliphatic compound that is extremely explosive. Nitroaromatic compounds, i.e., compounds containing one or more nitro (—NO₂) groups attached to an aromatic ring, are often explosive.

Typically nitroaromatic compounds with a plurality of nitro groups are more explosive than nitroaromatic compounds containing a single nitro group. Examples of nitroaromatic compounds utilized in munitions and other explosives include, but are not limited to, TNT (trinitrotoluene), RDX (1,3,5-trinitro-1,3,5-triazine), HMX (1,3,5,7-tetranitro-1,3,5,7-tetrazocane), tetryl(2,4,6-trinitrophenyl-N-methylnitramine), and C4 (an RDX explosive, which has been plasticized to be adhesive and malleable) (not shown), as shown in the structures below:

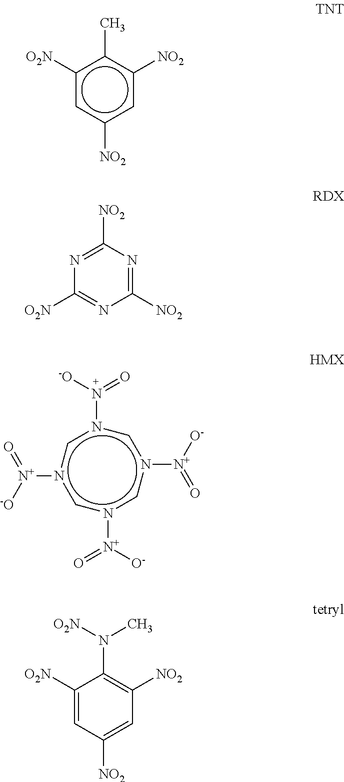

Thus, the present invention is directed to remediation of explosives generally, but more specifically explosives comprising nitro (—NO₂) groups. These compounds typically have the general formula provided below:

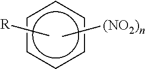

where R is hydrogen, nitrogen, or aliphatic, especially alkyl, and n=1-6, 1-4, or 1-3.

### B. Plant Absorption of Nitro-Based Explosives

In some embodiments, contaminated areas are seeded or over-seeded with plants that are capable of absorbing and sequestering nitro-based explosives from surface and near surface soils. As a result, any plant capable of absorbing a quantity of nitro-based compounds is suitable for use in practicing the present invention.

A number of plants have been shown to take up TNT and its metabolites, particularly 4-aminodinitrotoluene. These plants include, by way of example and without limitation, carrots, corn, grasses, Jimson weed, onions, sedges, tomatoes, winter wheat, cottonwood trees, and poplar trees, among others. Other nitro-based explosives also may be translocated from soil into plant foliage.

Exemplary plants include grasses belonging to the family Gramineae (also called Poaceae). Grasses typically are classified as either warm-season grasses or cool-season grasses. As the name implies, warm-season grasses grow primarily during hot weather and go dormant in cooler weather. In contrast, cool-season grasses begin their growth early in the spring and go dormant during hot, dry weather. Cool-season grasses begin growing again in the cooler autumn months if there is adequate water.

In particular embodiments, cool-season grasses are used to seed or over-seed the contaminated soil. Cool-season grasses grow well in nitrogen-rich soils, including soils contaminated with nitroaromatic compounds. Solely by way of example and without limitation, cool-season grasses include tall fescue (Festuca arundinacea), perennial ryegrass (Lolium perenne), orchard grass (Dactylis glomerata), among others, and particularly those grasses used in the dairy industry. The grasses used in the dairy industry are particularly tolerant to high levels of nitrogen. For instance, in modern dairy practice, about 500 units of nitrogen per acre are spread using the manure waste from dairies. Domestic lawns, when fertilized, use about 50 units of nitrogen per acre. If one were to use 500 units of nitrogen per acre, such as that used on a dairy, the high nitrate level would burn, or kill, the domestic lawn. Cool-season grasses have thick root mats that extend about two to five feet down into the soil and lesser rootlets that extend throughout the root mass and as far as five feet below the surface. This provides the grasses with a large root surface area for absorption.

In other embodiments, contaminated soil is seeded or over-seeded with warm-season grasses. For example, brome grasses (i.e., from the genus Bromus of the family Poaceae) are utilized. Alternatively, sedges, marshland plants from the family Cyperaceae, can be grown.

All of these grasses have been shown to take up nitroaryl and nitro-heteroaryl compounds (e.g., TNT and RDX, among others), from the soil and into their blades. Dairy grasses are, however, more effective than other grasses at taking up these compounds. Other nitro-based explosives also may be translocated from soil into plant foliage.

C. Microbes/Enzymes

The nitroaryl or nitro-heteroaryl compounds within the grass blades subsequently can be rendered nontoxic, or substantially nontoxic, to mammals by microbes found in the rumen of ruminant animals. Ruminants possess a complex stomach system having four compartments, the first and the largest of which is the rumen. The rumen is a highly reductive anaerobic environment in which a consortium of microbes metabolizes complex plant materials to make fatty acids and amino acids for utilization by the host. These anaerobic microbes also can reduce, or degrade, synthetic complex molecules, e.g., TNT, to benign or substantially benign nontoxic moieties. The hypothesis is that biotransformation reduces at least one nitro group to some other functional group, such as an amine. The reduced compounds can be incorporated into amino acids by microbes in the rumen.

The rumen is thought to include on the order of about one thousand different microorganisms, although very few (less than about 100) have been positively identified and named. The exact composition of the microbial consortium in the rumen may vary depending upon the species of the ruminant, its diet, and perhaps its geographical location.

The consortium of anaerobic microbes in the rumen includes both bacteria and archaea. Bacteria are single-cell, prokaryotic (i.e., without a nucleus) organisms. Archaea also are single-cell, prokaryotic organisms. However, archaea have a different evolutionary history and biochemistry than bacteria. For example, a group of anaerobic archaea known as methanogens aid in digestion and produce methane as a byproduct.

Anaerobic microorganisms also can be classified as facultative anaerobes or obligate anaerobes. Facultative anaerobes can survive in the presence or absence of oxygen. Facultative anaerobes perform aerobic respiration in the presence of oxygen. In the absence of oxygen, they perform anaerobic respiration or fermentation. Obligate anaerobes die when exposed to oxygen. Obligate anaerobes perform fermentation or anaerobic respiration.

Enzymes produced by at least some of the microbes within a ruminant animal's rumen degrade the munition compounds within the grass blades. For example, and without being bound by a theory of operation, some anaerobic microbes produce nitroreductases. Typically, a nitroreductase reduces a nitro (—NO₂) group to an amine (—NH₂) group. Thus, nitroreductases can react with a wide range of nitro-substituted compounds, such as nitro-aliphatic, nitroaryl, and nitro-heteroaryl compounds (e.g., nitrofurazones, nitroarenes, nitrophenols and nitrobenzenes, among others), including explosives such as TNT, RDX and GTN. Members of the nitroreductase family are flavoproteins that use NADPH/NADH as electron donors. Nitroreductase activity is widespread in nature, and the enzymes are produced constitutively, although their physiological role is poorly understood. Nitroreductases are distinguished on the basis of their ability to metabolize nitro-substituted compounds in the presence of oxygen, namely: Type I nitroreductase (oxygen-insensitive, i.e., active in the presence or absence of oxygen) and Type II nitroreductase (oxygen-sensitive, i.e., inactive in the presence of oxygen).

Nitroreductases reduce nitro-substituted compounds such as TNT, RDX and GTN into a series of metabolites. The initial steps in TNT metabolism are stepwise reduction of the nitro groups to amino groups. The last nitro group is reduced only under low oxidation-reduction potential (<−200 mV), making strict anaerobic conditions necessary for complete reduction of TNT into triaminotoluene or other unknown polar metabolites. For example, in an initial step, TNT may be converted to 4-amino-2,6-dinitrotoluene as shown below:

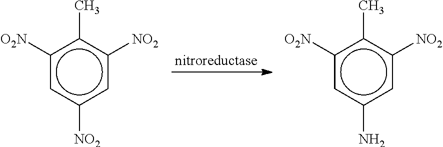

A more complete pathway of TNT reduction is shown below.

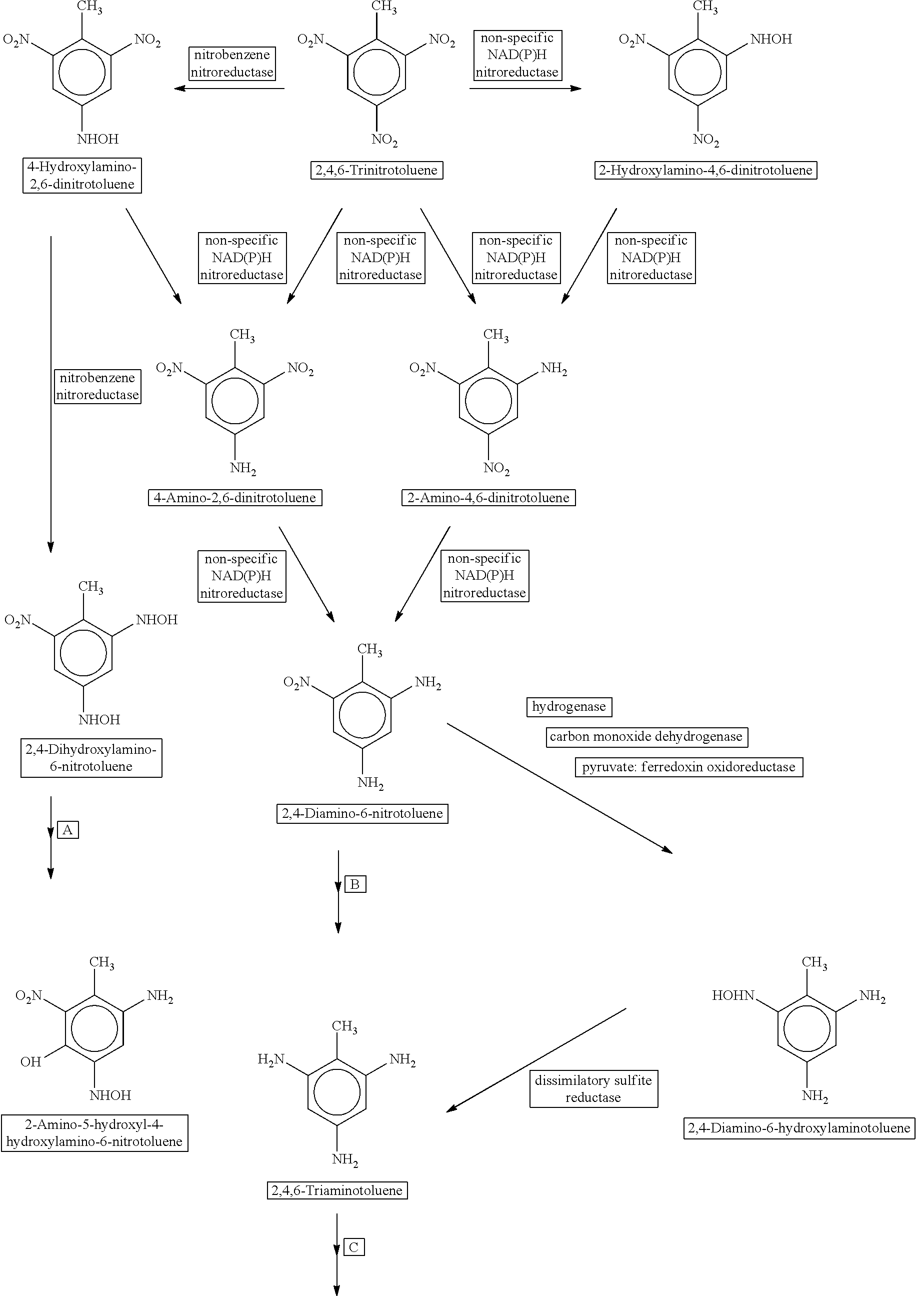

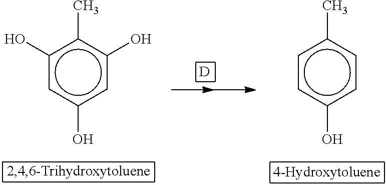

The TNT metabolites shown in the above pathway remain at least somewhat toxic until TNT is metabolized past 2,4,6-triaminotoluene. The reactions represented by A, B, C, and D are multi-step reactions as described in, respectively: Hughes et al., Environmental Science and Technology, 1998, 32:494-500; Ederer et al., J. Ind. Microbiol. Biotechnol., 1997, 18(2-3):82-8; Funk et al., Appl. Environ. Microbiol., 1993, 59(7):2171-7; ibid.

D. Contacting Nitro-Based Explosive Compounds with Microbes/Enzymes

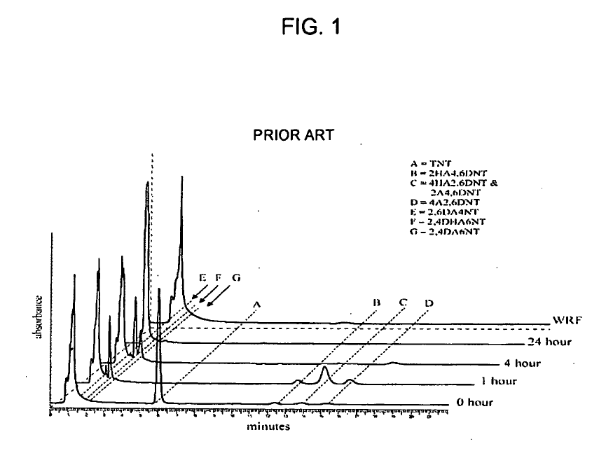

Previously, Fleischmann et al. investigated the degradation of TNT by bovine rumen fluid. (Biochem. and Biophys. Res. Comm., 314 (2004) 957-963.) Bovine rumen fluid was blended with McDougall's buffer (30:70), spiked with TNT and analyzed by HPLC at 0, 1, 4, and 24 hours after mixing. As shown in FIG. 1, TNT disappeared from the cultures within 1 hour. Reduction products initially formed included 2-hydroxyamino-4,6-dinitrotoluene (2HA4,6DNT), 4-hydroxyamino-2,6-dinitrotoluene (4HA2,6DNT), 2-amino-4,6-dinitrotoluene (2A4,6DNT), and 4-amino-2,6-dinitrotoluene (4A2,6DNT). Subsequently, diaminonitrotoluene and dihydroxyaminonitrotoluene products were formed, including 2,4-diamino-6-nitrotoluene (2,4DA6NT), 2,6-diamino-4-nitrotoluene (2,6DA4NT), and 2,4-dihydroxyamino-6-nitrotoluene (2,4DHA6NT). All known TNT degradation products were undetectable at 24 hours. WRF, as referred to in FIG. 1, is whole rumen fluid used as a negative control.

In other studies, triazine herbicides (simetryn, atrazine and propazine) were degraded by whole rumen fluid from sheep and cows in vitro under anaerobic conditions. The munition RDX belongs to the triazine family of molecules. Ruminal microbes in sheep also may be able to metabolize RDX in plant material in a rapid manner before the RDX is distributed systemically with potentially harmful effects. These microbes like produce, among other enzymes, nitroreductases and dehydrogenases.

1. Grazing

In exemplary embodiments of the present disclosure, after a period of plant growth during which the nitro-based explosives are translocated from soil and into the foliage, live ruminant animals, such as sheep, are introduced and allowed to graze on the plants. Grazing can continue for a preselected period and/or until the pertinent plants are ingested. Alternatively, grazing can be part of a rotation whereby, after a first period of time during which a particular plant or plants are allowed to grow, animals are purposefully grazed on the plant or plants for a second period of time. This cycle of plant growth followed by animal grazing may be repeated as long as plant growth continues. As the animals graze, enzymes produced by the microbes within each animal's rumen react with and degrade each contaminant and its metabolites.

In particular embodiments, a fencing system is used whereby the animals graze a section of land for a period of time and then are moved to a new section. For example, sheep may be allowed to graze on an area of land for about two to three days after which the sheep are moved to another area. The grazed area is allowed to “rest” for a period of time, such as about 25-30 days, during which the plants continue to grow and take up additional munitions compounds from the soil. This rest and rotation approach allows ranges and training lands to be available, for example, for about 27 days out of a 30-day period.

The strategy proposed here is a more practical and less costly agricultural solution using grazing plants that absorb munitions, such as a grass or grasses, and a particular ruminant, such as sheep, as compared to more traditional and costly engineering solutions. Further, this strategy offers an environmentally friendly solution to the problem of contaminated soils in that only plants and ruminant animals are employed to convert TNT and other munitions into nontoxic compounds, which are utilized by the animal for energy. No additional toxic by-products are formed, and minimal fuel and other mechanized equipment are involved. Additionally, humans have minimal contact with contaminated material, as the process relies on plants to absorb the munitions from the soil rather than requiring humans to excavate the material.

2. Administering Microbes to Other Animals

In other embodiments, an effective amount of anaerobic microbes capable of degrading remediable compounds, e.g., munitions compounds, can be obtained and administered by any suitable method to other ruminants lacking in endogenous ruminal microbes capable of degrading munitions compounds (e.g., deer, elk, rabbits, etc.). The anaerobic microbes can be obtained, for example and without limitation, from sheep or bovine ruminal fluid or from microbial cultures. The anaerobic microbes can be administered, for example, to the ruminants in a food product which the ruminants consume. One of ordinary skill in the art will understand that other routes of administration may be suitable. The ruminants then are allowed to graze on the plants which have absorbed the munitions compounds. The administered anaerobic microbes allow the ruminants to safely digest and degrade the plants containing the munitions compounds. By way of example and without limitation, the administered anaerobic microbes may include Butyrivibrio fibrisolvens nxy (ATCC #51255), Fibrobacter succinogenes S85 (ATCC #19169), Lactobacillus vitulinus T185 (ATCC #27783), Selenomonas ruminantium HD4 (ATCC #27209), Streptococcus bovis JB1 (ATCC #700410), Streptococcus caprinus 2.3 (ATCC #700065), Succinivibrio dextrinosolvens (ATCC #19716), or combinations thereof. One of ordinary skill in the art will understand that additional ruminal anaerobic microbes, not yet identified and named, also may be suitable for administration to ruminants to aid in safe digestion and degradation of plants containing munitions compounds.

### III. Microbe/Gene Isolation and Identification

The effect of TNT consumption on sheep rumen microbial populations was studied by feeding sheep TNT in the form of TNT-fortified grain supplements for a period of about three weeks. DNA was extracted from the rumen fluid of the sheep before and after the TNT administration, and a clone library was constructed from each of the rumen fluid samples using techniques known to a person of ordinary skill in the art. The DNA from the clone libraries was sequenced, and diversity indices were calculated. Phylogenetic trees also were constructed from the data. The study showed that feeding the sheep TNT for a 3-week period did not significantly change the overall microbial diversity in the rumen fluid. It is likely, however, that differences in diet will result in differences within the microbial population even though the overall microbial diversity remains substantially unchanged. For example, some microbes may disappear, while other microbes may appear within the population. Trends in richness values varied by indices and tended to increase.

Microbes involved in the degradation of remediable compounds, such as munitions compounds, can be isolated by obtaining and culturing a consortium of bacteria and archaea from ruminal fluid in an anaerobic environment. To enrich the culture with desired microbes and thus produce an enriched consortium, a medium is supplemented with one or more munitions compounds. For example, in some embodiments, the medium is supplemented with RDX to select for microbes involved in the degradation of RDX. This procedure encourages growth of microbes capable of RDX degradation. Ruminal fluid is added to the supplemented medium and incubated under anaerobic conditions.

After the enriched culture is prepared, colonies of individual isolates are obtained by methods known to one of ordinary skill in the art, such as anaerobic plate isolation where the medium used to prepare the plates has been supplemented with the desired munitions compound or compounds. Again, the supplementation selects for preferential growth of microbes capable of degrading the supplemented compound or compounds.

Individual isolates are identified by DNA sequencing and phylogenetic analysis using methods known to persons of ordinary skill in the art. DNA is extracted from the enriched culture and amplified by PCR, using primers designed to amplify universal archeal and bacterial genes, i.e., primers that can bind to conserved sequences within the genes. The PCR products are processed by denaturing gradient gel electrophoresis (DGGE) to separate the PCR products. Individual products are isolated from the gel and cloned into plasmids. Plasmid cultures then are grown, and the DNA is isolated from the plasmids. The isolated DNA is characterized by restriction fragment length polymorphism (RFLP) and sequencing. Phylogenetic analysis is used to identify and name the archaea and bacteria capable of degrading munitions compounds. The identified archaea and bacteria can then be used, for example and without limitation, for administration to other ruminants that lack endogenous microbes capable of degrading munitions compounds.

In other embodiments, another method for identifying the microbes capable of degrading munitions compounds includes anaerobically culturing ruminal fluid in a liquid medium supplemented with one or more radio-labeled munitions compounds. For instance, the use of RDX as a substrate by ruminal microbes can be determined by stable isotope probing (SIP). Liquid medium can be supplemented with [¹³C]RDX. As the microbes grow, they incorporate the carbon-13 into their nucleic acids.

Consumption of the [¹³C]RDX by microbes is confirmed with liquid chromatography/mass spectrometry (LC/MS). Samples of the liquid medium are taken at various time points during the incubation. LC/MS is used to determine the concentration of RDX molecules in the solution and to identify the presence of RDX metabolites in the solution. RDX breakdown is confirmed by a reduction in RDX concentration accompanied by an increase in the concentration of RDX metabolites. Additionally, mineralization of RDX to CO₂ can be confirmed using gas chromatography/isotope mass ratio spectrometry (GC/IMRS).

¹³C-labeled 16S RNA is separated from unlabeled nucleic acids on the basis of density using ultra-high speed centrifugation. 16S RNA is found in ribosomes and is selected for sequence analysis because the sequences are highly conserved within a species. The isolated 16S RNA is transcribed into cDNA using reverse transcriptase PCR (RT-PCR). The cDNA is subjected to DGGE and isolated from the gel.

The extracted DNA is cloned by the TOPO-TA (topoisomerase I cloning plasmid-terminal transferase) technique. TOPO-TA is available from Invitrogen. Vectors containing the cloned DNA are grown in culture, and the DNA is extracted and sequenced. Sequence analysis through established methods and databases is used to identify the microbes capable of degrading RDX.

In another study, the diversity of ruminal nitroreductase genes was assessed in sheep consuming various diets. A control sheep was fed a grass diet. Three sheep were fed a grass diet supplemented with TNT. An additional three sheep were fed a concentrate/foliage diet including soy, but without TNT. DNA was extracted from rumen fluid of the sheep. The DNA was amplified using touchdown polymerase chain reaction (PCR) techniques, as are known by persons of ordinary skill in the art. The primers used in the PCR were designed from known nitroreductase protein sequences. The known sequences were sorted into four groups based on a Bayesian phylogenetic tree (i.e., a diagram, or tree, showing the evolutionary relationships among various species), and a pair of degenerate primers was developed for each of Groups 1-4.

The PCR products then were cloned into vectors, amplified and sequenced by methods known to persons of ordinary skill in the art. The results showed that nitroreductases having sequences corresponding to the Group 1 primers were common in all the sheep, irrespective of diet. However, the sequence results from the Group 2 primers, which were found only in PCT products of sheep on grass diets (+/−TNT), indicated a closer match to dihydrodipicolinate reductase than to nitroreductase. Nitroreductases having sequences corresponding to the Group 3 primers were found only in sheep that consumed the concentrate/forage diet. Further sequence analysis was done with DOTUR (a computer program that takes a distance matrix describing the genetic distance between DNA sequence data and assigns sequences to operational taxonomic units (OTUs) using either the furthest, average, or nearest neighbor algorithms for all possible distances that can be described using the distance matrix). The analysis showed that the Group 3 nitroreductases actually had very low diversity, with only four clones indicated.

### IV. Examples

The following examples are provided to further illustrate certain features described above and are not meant to limit the disclosed methods to the particular embodiments disclosed.

### Example 1

### Uptake of TNT and RDX from Soil by Cool-Season Grasses

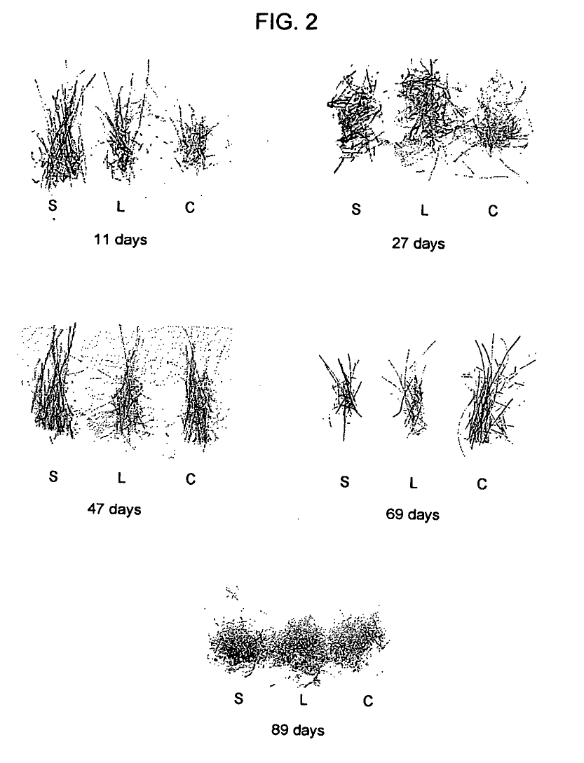

Initial studies using ¹⁴C-radiolabeled TNT showed that TNT was translocated from contaminated soils into the blades of cool-season grasses. Grasses were grown in three representative soil types: sand, loam and clay. The amount of ¹⁴C-labeled TNT in the plant tissue was measured by autoradiography. As shown in FIG. 2, successive grass cuttings from grasses grown in sand (S), loam (L), and clay (C) at 11 days, 27 days, 47 days, 69 days and 89 days after application of ¹⁴C-labeled TNT all showed translocation of radioactive residue into the plant foliage. The data indicates that up to 80% of TNT taken up by the plant is incorporated into the plant tissues. The remaining 20% is free TNT in the solutions of the plant, e.g., the sap. This study demonstrated that cool-season grasses have the capability to continuously extract and translocate nitroaryl compounds, such as TNT residues, from soil to new growth.

Field trials have shown that cool-season grasses, as well as native grasses, incorporate RDX into their plant tissues. The cool-season grasses, especially dairy grasses, had increased ability to take up RDX compared to the native grasses.

### Example 2

### Absorption and Distribution of TNT in Sheep

The purpose of this study was to determine how TNT is metabolized when consumed by sheep, its distribution throughout the sheep, and its excretion.

[¹⁴C]Toluene was purchased from Sigma Chemical Co., and 2,4,6-trinitro-[¹⁴C]toluene was synthesized and recrystallized from 95% ethanol to a radiochemical purity of 99.1% (8090±54 dpm/μg). Chemical purity was assessed by ¹H NMR, mass spectral analyses, and by HPLC with radiochemical detection for [¹⁴C]TNT or by UV detection (231 nm).

Three wether sheep (each 2 weeks old) were dosed with 35.5 mg each of dietary unlabelled TNT for 21 consecutive days. The TNT was administered in 0.5 kg of a grain supplement, i.e., ground corn. The supplement was prepared by sequentially adding aliquots of 2321 mg of TNT dissolved in 50 mL of acetone to 35.0 kg of cracked corn within a stainless steel ribbon mixer. Each acetone aliquot was allowed to evaporate and the grain was mixed for approximately 10 minutes prior to the addition of the next aliquot of TNT. The purpose of this feeding period was to enrich the population of ruminal microbes capable of breaking down TNT, such that it is comparable to the population of microbes in a sheep that regularly consumes TNT in its diet, because the microbial enrichment potentially changes the pathway of TNT degradation in sheep that repeatedly consume TNT.

On day 22, the sheep were orally dosed with 35.5 mg of U-ring labeled [¹⁴C]TNT (129 μCi, 99.1% purity) (i.e., all carbon atoms in the TNT were ¹⁴C). A total of 463.5 mg of [¹⁴C]TNT (1689 μCi) were dissolved in 5.0 mL of acetone; 0.383 mL of the acetone solution (129.4 μCi; 35.5 mg) were added to each of three gelatin capsules filled with cracked corn. The acetone was allowed to evaporate and each capsule was capped. At dosing, the three test sheep (weighing 41.9±3.0 kg) received a single capsule administered with a balling gun.

Blood, urine and feces were collected at regular intervals for 72 hours. At slaughter, tissues were quantitatively collected. Tissues and blood were analyzed for total radioactive residues; excreta were analyzed for total radioactive residues, bound residues, and TNT metabolites. The data are shown in Table 1 below:

TABLE 1

| | | wether | | | |
|---|---|---|---|---|---|
| fraction | item | 367 | 368 | 370 | average^{b} |
| urine | T0-6 | 16.8 | 13.7 | 11.5 | 12.6 |
| | T6-12 | 9.5 | 2.6 | 2.6 | 2.6 |
| | T12-18 | 3.2 | 0.8 | 0.6 | 0.7 |
| | T18-24 | 2.2 | 0.3 | 0.4 | 0.4 |
| | T24-32 | 0.5 | 0.2 | 0.2 | 0.2 |
| | T32-40 | 0.0 | 0.2 | 0.2 | 0.2 |
| | T40-48 | 0.7 | 0.1 | 0.2 | 0.2 |
| | T48-60 | 0.3 | 0.2 | 0.1 | 0.2 |
| | T60-72 | 0.4 | 0.1 | 0.1 | 0.1 |
| | total: | 33.6 | 18.2 | 15.9 | 17.1 |
| feces: | T0-6 | 0.2 | 0.2 | 0.2 | 0.2 |
| | T6-12 | 0.1 | 2.7 | 3.3 | 3.0 |
| | T12-18 | 0.1 | 11.4 | 12.1 | 11.8 |
| | T18-24 | 0.1 | 6.6 | 10.7 | 8.7 |
| | T24-32 | 0.0 | 11.7 | 15.8 | 13.8 |
| | T32-40 | 0.1 | 14.8 | 16.3 | 15.6 |
| | T40-48 | 0.0 | 9.4 | 8.6 | 9.0 |
| | T48-60 | 0.5 | 11.4 | 8.8 | 10.1 |
| | T60-72 | 3.9 | 5.8 | 3.3 | 4.6 |
| | total: | 5.0 | 74.0 | 79.1 | 76.6 |
| tissues | adipose | 0.00 | 0.00 | 0.00 | 0.00 |
| | kidney | 0.03 | 0.01 | 0.01 | 0.01 |
| | liver | 0.19 | 0.06 | 0.08 | 0.07 |
| | skeletal muscle | 0.47 | 0.00 | 0.03 | 0.02 |
| | bile | 0.01 | 0.00 | 0.00 | 0.00 |
| | blood | 0.00 | 0.00 | 0.00 | 0.00 |
| | bone | 0.24 | 0.00 | 0.05 | 0.03 |
| | brain | 0.00 | 0.00 | 0.00 | 0.00 |
| | eye | 0.02 | 0.01 | 0.00 | 0.01 |
| | heart | 0.01 | 0.00 | 0.00 | 0.00 |
| | large intestine content | 8.80 | 3.02 | 1.78 | 2.40 |
| | large intestine tissue | 0.14 | 0.02 | 0.01 | 0.02 |
| | lung | 0.03 | 0.00 | 0.01 | 0.01 |
| | rumen content | 51.18 | 3.84 | 1.36 | 2.60 |
| | rumen tissue | 0.93 | 0.09 | 0.03 | 0.06 |
| | skin | 0.38 | 0.06 | 0.08 | 0.07 |
| | small intestine content | 0.26 | 0.15 | 0.06 | 0.11 |
| | small intestine tissue | 0.08 | 0.02 | 0.01 | 0.02 |
| | spleen | 0.01 | 0.00 | 0.00 | 0.00 |
| | thyroid | 0.00 | 0.00 | 0.00 | 0.00 |
| | remainder of carcass | 0.31 | 0.03 | 0.02 | 0.03 |
| | total: | 63.09 | 7.31 | 3.53 | 5.33 |
| cage | | 0.4 | 0.2 | 0.4 | 0.3 |
| wash | | | | | |
| | total recovery (%): | 102.1 | 99.7 | 98.9 | 99.3 |

^{a}Data are expressed as a percentage of the [¹⁴C]TNT dose.
^{b}Average of wether 368 and 370.

Data collected from sheep #367 was disregarded. [Smith et al., Environ. Sci. Technol., 42 (2008) 2563-2569.] Plasma radioactivity peaked within one hour of dosing and was essentially depleted within 18 hours. Approximately 76% of the radiocarbon was excreted in feces, 17% in urine, with 5% being retained in the gastrointestinal tract and 1% retained in tissues. Parent TNT, dinitroamino metabolites, and diaminonitro metabolites were not detected in excreta. Ruminal and fecal radioactivity was essentially nonextractable using ethyl acetate, acetone, and methanol; covalent binding of fecal radioactive residues was evenly distributed among extractable organic molecules (i.e., soluble organic matter, soluble carbohydrate, protein, lipid, and nucleic acid fractions) and undigested fibers (cellulose, hemicellulose, and lignin). This study demonstrated that TNT reduction within the ruminant gastrointestinal tract leads to substantial immobilization of residues to organic matter. For example, the residues may be chemically bound to undigested fibers.

### Example 3

### Analysis of Ruminal Bacteria for TNT Degradation

Currently there are 22 known ruminal bacteria which are commercially available as purified cultures through the ATCC (American Type Culture Collection). These bacteria were analyzed for their potential to degrade TNT, 2-aminodinitrotoluene (2ADNT), 4-aminodinitrotoluene (4ADNT), 2,4-diaminonitrotoluene (2,4DANT), and 2,6-diaminonitrotoluene (2,6DANT).

The cultures were grown in a complex media with 40% clarified rumen fluid (per liter: 400 ml clarified rumen fluid; 2.0 g trypticase; 1.0 g yeast extract; 4.0 g cellobiose; 4.0 g sodium carbonate; 1.0 ml 0.1% resazurin; 10.0 ml VFA solution (concentration μmol/ml: 67.2 glacial acetic acid, 40.0 propionic acid, 20.0 butyric acid, 5.0 isobutyric acid, 5.0 2-methylbutyric acid, 5.0 valeric acid, 5.0 isovaleric acid); 0.3 g potassium phosphate, dibasic; 0.6 g sodium chloride; 0.3 g ammonium sulfate; 0.3 g potassium phosphate, monobasic; 0.08 g calcium chloride, dihydrate; 0.123 g magnesium sulfate, heptahydrate; 1.1 g sodium citrate, dihydrate). All media was dispensed in 9.7 ml aliquots into batch tubes prior to autoclaving; 0.2 ml reducing agent (1.25% cysteine sulfide) and 0.1 ml B-vitamins solution (per 100 ml: 20 mg thiamin HCl, 20 mg D-pantothenic acid, 20 mg nicotinamide, 20 mg riboflavin, 20 mg pyridoxine HCl, 1.0 mg p-aminobenzoic acid, 0.25 mg biotin, 0.25 mg folic acid, and 0.1 mg cyanocobalamin) was added prior to inoculation. TNT was added to the cultures to a final concentration of 30 mg/L. Cultures were grown at 39° C. with shaking (150 rpm) for 18-24 hours between transfers. Cultures were transferred at least twice before degradation kinetic experiments to insure actively growing cells were used. Experiments were conducted in a Coy anaerobic glovebox (Coy Inc., Grass Lake, Mich.) with H₂ gas level at 7-8%, the remaining gas was CO₂. A time course sampling was done hourly for six hours with a final sampling time point at 24 hours to confirm earlier results. Cultures were grown in microtiter plates (Becton Dickinson Labware, Franklin Lakes, N.J.) in 200 μl volumes.

2,4,6-Trinitrotoluene (TNT), 4-amino-2,6-dinitrotoluene (4-ADNT), and 2-amino-4,6-dinitrotoluene (2-ADNT) were purchased from Chem Service (West Chester, Pa.). 2,4-Diamino-6-nitrotoluene (2,4-DANT) and 2,6-diamino-4-nitrotoluene (2,6-DANT) were purchased from AccuStandard (New Haven, Conn.). Solvents were HPLC grade and were purchased from Fisher Scientific (Tustin, Calif.). Reagents were of analytical grade and were purchased from Sigma-Aldrich (St. Louis, Mo.). An ELGA Ultra PureLab (ELGA Inc., Cary, N.C.) reverse osmosis water purification system was used to generate Milli-Q (resistance>18.2 MΩ-cm) quality water for all aqueous solutions.

HPLC analyses were carried out by a modification of the method described by Khan et al. [Journal of Industrial Microbiology and Biotechnology, 18 (1997) 198-203.] Separations were performed using a guard column hand packed with Pellicular C8 material and a Nova-Pak C8 analytical column (150 mm×3.9 mm id, 4 μm particle size, Waters, Milford, Mass.). The column was eluted under isocratic conditions with water and 2-propanol (82:18) at a flow rate of 1 ml/min with a total run time of 24 min. The HPLC system consisted of a Perkin-Elmer Series 200 Pump equipped with a Perkin-Elmer ISS 200 autosampler and photodiode array detector (Perkin-Elmer Series 200) monitoring at 230 nm. TotalChrome software (Perkin-Elmer) was used to analyze and quantify HPLC data.

The results of the analysis are shown in Table 2.

TABLE 2

| Substrate | Organism | Degrades | TNT | 2ADNT | 4ADNT | 2,4ADNT | 2,6ADNT | Summ mel | SUM ALL |
|---|---|---|---|---|---|---|---|---|---|
| S | Lactobacillus vitulinus T185 | +++ | 0% | 0% | 0% | 0% | 0% | 0% | 0% |
| S | Succinivibrio dextrinosolvens | +++ | 0% | 0% | 0% | 0% | 0% | 0% | 0% |
| A | Selenomonas ruminatium HD4 | +++ | 0% | 0% | 0% | 0% | 2% | 2% | 2% |
| C | Fibrobacter succinogens S85 | +++ | 3% | 0% | 0% | 0% | 0% | 0% | 3% |
| S | Streptococcus caprinus 2.2 | +++ | 5% | 0% | 6% | 9% | 4% | 19% | 24% |
| W | Eubacterium ruminentium GA195 | +++ | 0% | 7% | 20% | 0% | 0% | 27% | 27% |
| S | Streptococcus bovis IFO 12057 | +++ | 9% | 0% | 12% | 1% | 9% | 22% | 31% |
| S | Lactobacillus ruminis ATCC 20403 | +++ | 0% | 18% | 16% | 0% | 0% | 34% | 34% |
| W, (C) | Butyrivbrio fibrosolvens nxy | +++ | 0% | 24% | 8% | 8% | 2% | 42% | 42% |
| S | Clostridium pasteurianum 5 | +++ | 1% | 2% | 22% | 0% | 22% | 45% | 46% |
| C | Ruminococcus albus 8 | +++ | 1% | 32% | 13% | 0% | 0% | 45% | 46% |
| A | Wolinella succinogens ATCC 29543 | +++ | 0% | 28% | 19% | 0% | 12% | 59% | 59% |
| S | Ruminobacter amytophilus ATCC 29744 | +++ | 0% | 23% | 37% | 0% | 0% | 60% | 60% |
| C | Clostridium polysaccharolyticum ATCC 33142 | ++ | 22% | 20% | 19% | 0% | 0% | 38% | 60% |
| S, W, P | Prevotella rumincola GA33 | ++ | 23% | 0% | 41% | 0% | 0% | 41% | 64% |
| A | Megashera elsdenii T-81 | ++ | 17% | 5% | 37% | 0% | 11% | 52% | 69% |
| S, W, P | Prevotella bryantii B1 4 | +− | 26% | 26% | 18% | 0% | 0% | 44% | 70% |
| C | Ruminococcus flavenflaciens C94 | ++ | 16% | 10% | 52% | 0% | 0% | 62% | 78% |
| S | Treponema bryantii ATCC 33254 | + | 35% | 4% | 14% | 0% | 0% | 18% | 53% |
| S, W, P | Prevotella albensis | + | 41% | 22% | 13% | 0% | 0% | 35% | 76% |
| A | Desulfovibrio desulfuricans ssp dosulluricans A | + | 43% | 21% | 34% | 0% | 0% | 56% | 99% |
| L | Anaerovibrio lipolytica 7553 | − | 98% | | | | | | |

+++: >90% degradation
++: 80-90% degradation
+: 50-80% degradation
(+): 20-5O % degradation
(−): <20% P Protein
−: no degradation
C Cellulose
S Starch/sugar
A Acids/secondary metabolites
W Cell walls
L lipids

The results clearly showed that several microbes within the rumen are capable of degrading TNT and its metabolites.

The rate and extent of TNT biodegradation then were studied with seven of the known ruminal bacteria that had been found to degrade more than 90% of 100 mg/L TNT in 24 hours and showed the ability to transform the monoamino metabolites, e.g., 2-amino-4,6-dinitrotoluene and 4-amino-2,6-dinitrotoluene. These ruminal bacteria are listed below in Table 3.

TABLE 3
Bacterial strains used for degradation kinetic experiment

| Organism Name | ATCC # |
|---|---|
| Butyrivibrio fibrisolvens nxy | 51255 |
| Fibrobacter succinogenes S85 | 19169 |
| Lactobacillus vitulinus T185 | 27783 |
| Selenomonas ruminantium HD4 | 27209 |
| Streptococcus bovis JB1 | 700410 |
| Streptococcus caprinus 2.3 | 700065 |
| Succinivibrio dextrinosolvens | 19716 |

For kinetic experiments, the same media, methods, and analyses were utilized, but eight concentrations of TNT were used: 0, 10, 20, 30, 40, 50, 75, 100 mg/L. Cultures were transferred at least twice before degradation kinetic experiments to insure actively growing cells were used. Within each plate, each concentration of TNT Was done in triplicate along with the controls of media with TNT, but with no bacteria. At each time point, there were three plates that were sacrificed for analysis. Time from culture addition to the 0 hr sample was approximately 30 min. Experiments where complete degradation of the lower levels of TNT occurred before the 0 hr samples were repeated with half of the culture inoculum used in the first experiment. Triplicate samples within a plate were pooled; a 10 μl sample was removed for bacterial cell concentration determination, the remaining sample was centrifuged for 5 minutes at 16,000×g and ran by HPLC. B. fibrisolvens culture resulted in a viscous fluid even after centrifuging, so the samples were extracted twice in equal volumes of ethyl acetate. Ethyl acetate was evaporated under nitrogen gas, and then the sample was reconstituted in 50 μl methanol and 450 μl Milli-Q quality water. Standards were extracted at the same time for HPLC quantitation.

Bacterial concentrations for the 0 hr sample were determined by direct counting using a Petroff-Hausser Counter for sperm and bacteria (Hausser Scientific Partnership, Horsham, Pa.) following manufacturer's protocol.

The following equation was used to calculate the biodegradation constant and the Michaelis-Menten constant.

$$\begin{matrix}{R}_{S 0} = \frac{k * {X}_{0} * {S}_{0}}{{K}_{m} + {S}_{0}} \\ or & ( 1 ) \\ {R}_{X0} = \frac{{R}_{S 0}}{{X}_{0}} = \frac{{kS}_{0}}{{K}_{m} + {S}_{0}} & ( 2 )\end{matrix}$$

Where R_{S0} is the initial rate of TNT degradation (mg TNT 1⁻¹ h⁻¹), S₀ is the initial TNT concentration (mg L⁻¹), Kₘ is the Michaelis-Menten constant, X₀ is the initial biomass concentration (10⁶ cells L⁻¹), k is the biodegradation constant for TNT (h⁻¹). R_{S0} is determined by using the initial TNT degradation rate as R_{S0}=ΔS/Δt, where ΔS is (S₀-S₁) and Δt is 1 h. R_{X0} is the specific rate of TNT degradation (mg TNT 10⁶ cells⁻¹ h⁻¹) and is determined as R_{S0}/X₀ in a double reciprocal form Eq. 2 can be written as follows:

$$\frac{1}{{R}_{X 0}} = \frac{1}{k} + \frac{{K}_{m}}{k} \frac{1}{{S}_{0}} \qquad ( 3 )$$

A plot of 1/R_{X0} versus 1/S₀, or Lineweaver-Burk plot, yields a line with a slope of Kₘ/k and an intercept of 1/k, where k is the biodegradation constant (calculated per million cells, units of h⁻¹) and Kₘ is the Michaelis-Menten constant (calculated per million cells, units of mg L⁻¹). Inhibition was tested by plotting the specific rate of degradation versus the initial TNT concentration. A linear line indicated no inhibition and a curved line indicated inhibition. When inhibition was detected the concentrations that formed the linear portion of the line was used in the Lineweaver-Burk plot for calculations.

All organisms tested demonstrated Michaelis-Menten kinetics with a linear relationship on the Lineweaver-Burk plot. Controls without bacteria had less than 15% removal of TNT with the primary metabolite recovered being 4-ADNT and a lower level of 2-ADNT, total recovery was >90% of initial TNT (data not shown).

Superior results were found with B. fibrisolvens nxy and Suc. dextrinosolvens with biodegradation rate constants of 5.63 and 11.39 per 10⁶ cells, respectively. The biodegradation constants of all seven bacteria are shown in Table 4 below:

TABLE 4

| Organism | K (h⁻¹) |
|---|---|
| B. fibrisolvens nxy | 5.63 |
| F. succinogenes S85 | 0.49 |
| L. vitulinus T185 | 1.75 |
| Sel. ruminantium PC-18 | 2.34 |
| Strep. bovis JB1 | 0.31 |
| Strep. caprinus 2.2 | 0.74 |
| Suc. dextrinosolvens | 11.39 |

### Example 4

### Influence of TNT on Sheep Rumen Bacterial Populations

The purpose of this study was to determine whether consuming TNT as part of the daily diet would affect the diversity of the sheep rumen bacterial population. Whole rumen fluid was collected via a stomach tube from two yearling wether sheep before the start of treatment and after 21 days of treatment. The sheep received approximately 33.5 mg TNT in TNT-fortified grain supplements per day for three weeks.

The animals used in the study did not exhibit signs of TNT toxicity. The methods and excretory data were similar to that of Example 2, with a majority of the TNT excreted in the feces (76.6%) as bound residues or in the urine (17.1%) as unknown polar metabolites. No toluene ring structures were detected in the extraction data, indicating that TNT was metabolized in the rumen prior to absorption and the majority of the metabolites formed were bound to the digestive contents.

DNA was extracted from each of the four rumen fluid samples using the Puregene kit (Gentra Systems, Minneapolis, Minn.) following the body fluid protocol. Using techniques known to persons of ordinary skill in the art, a clone library was generated from the DNA from each rumen fluid sample. Fifty clones from each library were sequenced. Several diversity indices were calculated using DOTUR. See Table 5.

TABLE 5
Diversity and richness estimates for libraries constructed from
sheep rumen fluid before and after consuming TNT for 21 days

| Library^{a} | OTU | Shannon-Wiener^{b} | Chao 1^{b} | Ace^{b} |
|---|---|---|---|---|
| AN2 | 35 | 3.48 | 54 | 63 |
| Pre-TNT | | (3.28-2.67) | (42-89) | (55-72) |
| AN2 | 33 | 3.24 | 76 | 145 |
| Post-TNT | | (2.96-3.52) | (48-152) | (76-330) |
| AN3 | 31 | 3.22 | 94 | 91 |
| Pre-TNT | | (2.97-3.46) | (52-225) | (53-193) |
| AN3 | 31 | 3.20 | 82 | 97 |
| Post-TNT | | (2.95-3.46) | (48-181) | (55-207) |

^{a}Sheep numbers are AN2 and AN3, with one library before TNT exposure (Pre-TNT) and one after TNT exposure for 21 days (Post-TNT)
^{b}95% confidence intervals are in parentheses

Bayesian statistics were used to estimate the relationships and sequence continuity of the clones, and to construct phylogenetic trees, as shown below in Trees 1 and 2.

Feeding the sheep TNT for a period of three weeks did not significantly change the overall microbial diversity in the rumen fluid. Trends in richness values varied by indices and tended to increase. However, the sample size was too low to accurately estimate species richness. The results do indicate that consuming low levels of TNT did not adversely affect the rumen microbial populations.

### Example 5

### Isolation of Ruminal Microorganisms Capable of RDX Degradation

Both archaeal and bacterial species are thought to be involved in RDX degradation in the rumen. The following techniques were used to isolate and identify these organisms.

Rumen fluid obtained from three sheep was pooled in a pre-warmed thermos, in a manner consistent with maintaining an anaerobic environment. [D. Wachenheim, Veterinary and Human Toxicology, 1992, 34(6):513-17.] Anaerobically prepared 3-mL tubes of various types of media each were inoculated with one milliliter of rumen fluid in an anaerobic glove box. The types of media included a) complex media, b) easy media, c) media-E, d) low basal nitrogen media, and e) methanogenic media, the compositions of which are shown below:

| | Complex | | Easy | | Low Basal | |
|---|---|---|---|---|---|---|
| Ingredient | (per L) | | (per L) | | Nitrogen | |
| Trypticase | 2.0 | g | 2.0 | g | 20 | |
| Yeast Extract | 1.0 | g | 1.0 | g | | |
| Cellobiose | 4.0 | g | 4.0 | g | | |
| Mineral Solution 1 | 25.0 | ml | 25.0 | ml | | |
| Mineral Solution 2 | 25.0 | ml | 25.0 | ml | | |
| VFA solution | 10.0 | ml | 10.0 | ml | | |
| Clarified Rumen Fluid | 400 | ml | | — | — | |
| Resazurin (0.1%) solution | 1.0 | ml | 1.0 | ml | | |
| Sodium Carbonate, Na₂CO₃ | 4.0 | g | 4.0 | g | 25 | |
| Distilled water | 519 | ml | 919 | ml | | |
| Agar | | | | | | |
| Dextrose | | | | | | |
| | Per 10 ml | | Per 10 ml | | Per 10 ml | |
| B-vitamins^{a} | 0.1 | ml | 0.1 | ml | 0.1 | ml |
| 1.25% Cysteine sulfide^{a} | 0.2 | ml | 0.2 | ml | 0.2 | ml |

30
^{a}Added as a sterile addition after autoclaving prior to inoculation

### Media-E:

| Component | Grams per Liter |
|---|---|
| NH₄Cl | 1.0 |
| Na₂SO₄ | 1.0 |
| CaCl₂•2H₂O | 0.68 |
| MgCl₂•6H₂O | 1.77 |
| Sodium Lactate (d = 1.31 g/mL) | 2.67 mL |
| Yeast Extract | 1.0 |
| FeSO₄•7H₂O | 0.5 |
| Resazurin (1% w/v) | 1.0 mL |
| *Ascorbic Acid (free acid) | 0.1 |
| *Thioglycollic Acid (Na Salt) | 0.1 |

*Add after gassing for 20 min.

### Methanogenic Media:

| Component | 300 ml | |
|---|---|---|
| trypticase | 1.2 | g |
| yeast extract | 0.6 | g |
| MW #1 (2x)¹ | 11.25 | ml |
| MW #2 (2x)² | 11.25 | ml |
| sodium acetate | 0.6 | g |
| sodium formate | 0.6 | g |
| 0.1% FeSO₄•(aq) | 0.3 | ml |
| clarified rumen fluid | 1.5 | ml |
| sodium carbonate | 1.2 | |
| deionized water | 253.2 | ml |

*After autoclaving, 0.2 ml 1% cysteine sulfide and 0.1 ml B vitamins are added to each 9.7 ml aliquot of methanogenic media.
¹MW #1: 3.0 g potassium phosphate dibasic and 1.0 g sodium citrate dihydrate dissolved in deionized water to 500 ml.
²MW #2: 6 g NaCl, 6 g (NH₄)₂SO₄, 3 g KH₂PO₄, 0.795 g CaCl₂•H₂O, 1.23 g
MgSO₄•H₂O, and 10.0 g Na₃C₆H₅O₇•2H₂O dissolved in 250 ml deionized water and diluted to 500 ml.

RDX was added to each culture to achieve a final concentration of 33 μg/mL RDX. Positive controls were prepared by adding RDX to media without the addition of rumen fluid. Because RDX is light-sensitive, the cultures and controls were incubated in the dark at 39° C. on a rotary shaker at 150 rpm. Time-point samples were taken at inoculation (0 hour) and every 24 hours for up to one week and processed for HPLC analysis.

HPLC analyses were performed using a Dionex, Acclaim Explosives E1 column (250×4.6 mm, 5 μ) with a Phenomenex Security Guard Cartridge, C8. The column was run at a flow rate of 1.0 mL/min. at 32-32.2° C., using an injection volume of 10 μl. The mobile phase was methanol/water (43:57) with a total run time of 30.5 min. The HPLC system consisted of a Perkin-Elmer Series 200 LC Quaternary Pump equipped with a Perkin-Elmer Series 200 autosampler and a Perkin-Elmer series 200 UV/VIS detector monitoring at 254 nm. RDX eluted at approximately 8.47 minutes. Rumen fluid samples were prepared by mixing 0.5 ml rumen fluid and 0.5 ml acetonitrile. The mixture was centrifuged at 15,000 rpm, and the supernatent was injected.

After at least three transfers to fresh media with reducing agent, vitamins, and RDX at three-day intervals, DNA was extracted from cultures capable of RDX degradation in 7 days or less and used for polymerase chain reaction (PCR), cloning, and sequencing.

The extracted DNA underwent PCR with primers designed to amplify universal archeal and bacterial genes. [Yu et al., Biotechnol. Bioeng., 89 (2005) 670-679; Weisburg et al., J. Bacteria., 173 (1991) 697-703.] The gene products obtained from PCR were subjected to denaturing gradient gel electrophoresis (DGGE).

DNA bands isolated from the DGGE will be used for cloning and plasmid preparations. The results will be analyzed using restriction fragment length polymorphisms (RFLP), sequencing and phylogenetic analysis

Colonies of individual bacteria and archaea can be isolated from the RDX enrichments by streaking samples of the enriched culture onto anaerobic plates wherein the media used to prepare the plates is supplemented with RDX to encourage growth of microbes capable of degrading RDX.

### Example 6

### Identification of Microbes Capable of Using RDX as a Carbon Source

The use of RDX as a substrate by ruminal microbes can be determined by stable isotope probing. Stable isotope probing includes providing the microbes with a food source containing carbon-13, e.g., [¹³C]RDX. Only microbes capable of degrading RDX will use it as a carbon source.

Carbon-13 labeled RDX was acquired from Cambridge Isotopes. Labeled RDX was added to ovine ruminal fluid and incubated under anaerobic conditions. As the microbes consumed the labeled RDX and multiplied, the microbes formed ¹³C-labeled nucleic acids. Breakdown of RDX in the media by microbes was confirmed by liquid chromatography/mass spectrometry (LC/MS), which was used to determine the concentration of RDX molecules in the solution and to identify the presence of RDX metabolites in the solution.

RDX is detected in mass spectrometry as an acetate adduct with a molecular weight of 281. The acetate is derived from the mobile phase of the HPLC system. Operating conditions for the mass spectrometer (3200 QTRAP LC-MS/MS/MS from Applied Biosystems) were optimized by direct infusion of ¹²C-RDX or ¹³C-RDX using Analyst software, in negative mode using an APCI probe. HPLC settings were applied from a previous publication on quantification of explosives by LC-MS. Multiple reaction monitoring (MRM) was selected as the MS scan type to quantify the amount of RDX present in the sample. A transition of 281→46 was chosen for this. A standard curve from 10 ng/ml-100 ng/ml ¹²C-RDX or ¹³C-RDX was created against which the samples were compared to determine their concentration.

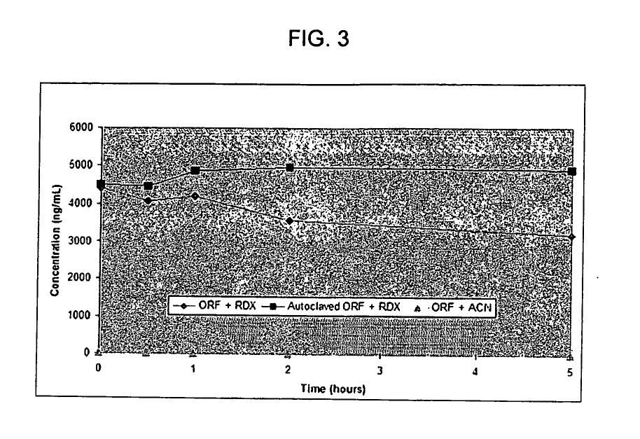

As shown in FIG. 3, a 27% degradation of 60 mg/L RDX was observed over five hours in ovine ruminal fluid. A positive control of autoclaved ovine ruminal fluid plus 60 mg/L RDX showed no degradation over 5 hours. A negative control of ovine ruminal fluid and acetonitrile showed no RDX. Information-dependent acquisition (IDS) experiments were set up to pick out new metabolites formed in the sample incubations. An MRM survey scan selectively pulled out any RDX metabolites which were then further characterized using an enhanced product ion scan. Mineralization of RDX to CO₂ can be confirmed by gas chromatography/isotope ratio mass spectrometry (GC/IRMS).

Using techniques known to persons of ordinary skill in the art, ultra-high speed centrifugation was used to separate the nucleic acids on the basis of density. ¹³C-labeled 16S RNA was transcribed into cDNA using reverse transcriptase PCR (RT-PCR. The resulting cDNA was subjected to denaturing gradient gel electrophoresis. Novel banding patterns of cDNA (i.e., bands not seen in DNA from cultures grown in the absence of RDX) are extracted from the gel using conventional techniques.

The extracted bands then can be subjected to TOPO-TA cloning, a technique in which DNA fragments are amplified in the presence of TA (a terminal transferase), which adds a single 3′-A overhang to the end of each strand. Topo-TA is commercially available from Invitrogen. The amplified DNA fragments can be cloned into TOPO (topoisomerase I) vectors, i.e., vectors having a 5′-(C/T)CCTT-3′ sequence at each end and capable of ligating the amplified DNA fragments into the vector using a topoisomerase enzyme.

Vector cultures then can be grown, and DNA isolated from the vectors. The DNA can be sequenced, and sequence analysis through established methods and databases can be used to identify the specific microbes capable of degrading RDX.

### Example 7

### Diversity of Nitroreductase Genes in Sheep Rumen Under TNT-Supplemented and Concentrate Diets

The purpose of this study was to determine differences in ruminal nitroreductase genes in sheep consuming various diets. A control sheep was fed a grass diet without TNT (TNT−). Three sheep were fed a grass diet supplemented daily with 33.1 mg TNT in 0.5 kg ground corn (TNT+). The three remaining sheep were fed a concentrate/forage diet without TNT. After about seven days, whole rumen fluid was obtained from each sheep.

Primer sets were designed to amplify nitroreductases from the sheep rumen. Seventeen complete nitroreductase protein sequences from the NCBI (National Center for Biotechnology Information) database were obtained and aligned using ClustalW. Based on this data, a Bayesian phylogenetic tree was constructed, and the sequences were sorted into four groups, as shown below in Table 3.

TABLE 3

| Group I | Group 2 | Group 3 | Group 4 |
|---|---|---|---|
| Bartonella henselae | Enterobacter | Clostridium perfringens | Lactobacillus casei |
| | sp. 638 | str. 13 | ATCC 334 |
| Burkholderia cepacia | Shewanella | Streptococcus pyogenes | Vibrio harveyi |
| AMMD | sp. MR 4 | MGAS8232 | |
| Pseudomonas putida | Rhodopseudomonas | Enterobacter cloacae | Escherichia coli NfsA |
| PnbA | palustris BisA53 | | |
| Acidovorax | | Escherichia coli NfsB | Salmonella typhimurium |
| sp. JS42 | | | |
| Pseudomonas | | | |
| pseudoalcaligenes | | | |

The CODEHOP program was used to develop a set of degenerate primers for each of the four groups, the sequences of which are shown below:

Group 1^{a}:
Forward Primer:
5′-CCCGACCGGCACCAACMYBCARCCNTG mybcarccntg-3′
Reverse Primer:
5′-TCGGCCCAGCCCAGVSHCATRCC agvshcatrcc-3
Group 2:
Forward Primer:
5′-GCCGGGATGCGGGTNCNNGAYCAYG nccngaycayg-3′
Reverse Primer:
5′-GGAGTCCCTATGTACACGAAACCCRCDATYTKNTC
rcdatytkntc-3′
Group 3:
Forward Primer:
5′-CCTTCTTCTTTTAATTTACAACCATGGMAWTTTDT
tggmawtttdt-3′
Reverse Primer:
5′-CTTTATCATAATCAAATCCTTCTATAGGACAWGHATC
ggacawghatc-3′
Group 4:
Forward Primer:
5′-TCCCACTTCCTGCAGTGCTKGWSBAT tgctkgwsbat-3′
Reverse Primer:
5′-TGCTGCGGCATGCGNGGYTTRAWNT gnggyttrawnt-3′
^{a}The capital letters in each sequence indicate
the 5′ clamp region. Lower case letters indicate
the 3′ degenerate region.

The rumen fluid samples from the sheep consuming each type of diet were combined, and genomic DNA was extracted from the rumen fluid samples using published methods. Touchdown PCR was performed to amplify the nitroreductase gene products. Touchdown PCR is a variant of PCR in which the annealing temperature is incrementally decreased in each cycle. As a result, the first sequence amplified is the one between the regions of greatest primer specificity and is most likely to be the sequence of interest. Amplification of nonspecific sequences is reduced with this technique. Fourteen cycles were performed at 64-57° C., followed by amplification at 57° C. for 15 cycles. Of the four primer sets, only two sets (Groups 1 and 2) generated products for the TNT− and TNT+ samples. A different primer set (Group 3) amplified products in the DNA from the concentrate/forage population.

The PCR products were cloned and sequenced. However, cloning was unsuccessful with the PCR products from the Group 1 primers. The PCR products from the group 2 primers were successfully cloned, but the resulting sequence data indicated the nearest match was dihydrodipicolinate reductase, not nitroreductase. Sequence data from the cloned group 3 PCR products was translated to protein sequences using the translate tool at Expasy. The sequences were then aligned using Mesquite. A Bayes block was constructed using the BLOSUM amino acid model. The alignment was run for 500,000 generations with a sampling frequency of 10 and a burn-in set at 37,500. BLAST hits of the protein sequences were included in constructing a phylogenetic tree using a Bayesian approach. Consensus trees were viewed in TreeView X. See Tree 3 below. TreeClimber was used to identify unique nitroreductase populations between the three test groups.

Additionally, nucleotide sequences obtained from the touchdown PCR were aligned using ClustalW. A DNA distance matrix was made using DNAdist. DOTUR analysis was performed on the sequences.

The results showed that nitroreductase diversity was affected by feed type. Group 1 nitroreductases appeared to be common in the sheep, irrespective of diet. Group 3 nitroreductases preferentially were found in sheep consuming a concentrate/forage diet. DOTUR analysis at the 95% confidence level suggested the diversity of Group 3 nitroreductases was very low, with only four clones indicated.

In view of the many possible embodiments to which the principles of the disclosed invention can be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for remediation of remediable compounds, including nitro-based explosives, reaction products thereof, and/or metabolites thereof, comprising:
identifying areas of land having soil containing the remediable compounds;
growing, or encouraging the growth of, plants within the soil for a first period of time, wherein the plants are capable of absorbing and sequestering the remediable compounds from the soil; and
rendering the remediable compounds substantially nontoxic to mammals by exposing the plants to anaerobic microbes capable of degrading the remediable compounds for a second period of time.

2. The method of claim 1 where the remediable compounds are nitroaliphatic compounds, nitroaryl compounds, nitro-heteroaryl compounds, or combinations thereof.

3. The method of claim 1 where the plants comprise one or more varieties of cool-season grasses.

4. The method of claim 3 where the one or more varieties of cool-season grasses comprise tall fescue, perennial ryegrass, orchard grass, or combinations thereof.

5. The method of claim 1 where the anaerobic microbes are obligate anaerobes.

6. The method of claim 1 where the anaerobic microbes are ruminal microbes.

7. The method of claim 6 where the ruminal microbes are sheep ruminal microbes.

8. The method of claim 6 where the ruminal microbes comprise Butyrivibrio fibrisolvens nxy, Fibrobacter succinogenes S85, Lactobacillus vitulinus T185, Selenomonas ruminantium HD4, Streptococcus bovis JB1, Streptococcus caprinus 2.3, Succinivibrio dextrinosolvens, or combinations thereof.

9. The method of claim 6 where the ruminal microbes are bovine ruminal microbes.

10. The method of claim 1 where exposing the plants to the anaerobic microbes comprises having at least one ruminant animal consume the plants to expose the remediable compounds to microbes in the animal's rumen.

11. The method of claim 10, wherein the ruminant animal lacks endogenous microbes capable of degrading the remediable compounds, further comprising administering to the ruminant animal an effective amount of anaerobic microbes capable of degrading the remediable compounds.

12. The method of claim 11 where administering an effective amount of anaerobic microbes comprises providing a food product containing the anaerobic microbes to the ruminant animal, wherein the ruminant animal consumes the food product.

13. The method of claim 1 where the first period of time is about 25 to 30 days and the second period of time is about 2 to 3 days.

14. The method of claim 1 where the remediable compounds are nitroaromatic compounds and the nitroaromatic compounds are degraded by nitroreductases produced by the anaerobic microbes.

15. The method of claim 1 where the remediable compounds are degraded to benign or substantially benign compounds.

16. An enriched consortium of bacteria and archaea in the rumen of a ruminant animal, wherein the enriched consortium comprises different bacteria and archaea than an unenriched consortium, and wherein the enriched consortium was produced by consumption of nitroaliphatic compounds, nitroaryl compounds, nitro-heteroaryl compounds, or combinations thereof by the ruminant animal.

17. The enriched consortium of claim 16 where the consortium is capable of using the nitroaliphatic compounds, nitroaryl compounds, nitro-heteroaryl compounds, or combinations thereof as a carbon substrate.

18. A method for isolating and identifying anaerobic microbes capable of degrading a remediable compound, including a nitro-based explosive, reaction product thereof, and/or metabolite thereof, comprising:
obtaining a consortium of anaerobic microbes;
encouraging the growth of anaerobic microbes within the consortium that are capable of degrading the remediable compound, where encouraging the growth comprises exposing the consortium for a period of time to a medium comprising the remediable compound;
after the period of time, isolating an individual anaerobic microbe from the consortium;
amplifying and extracting DNA from the individual microbe;
obtaining a DNA sequence of at least a portion of the DNA; and
using the DNA sequence to identify the individual microbe.

19. The method of claim 18, further comprising administering an effective amount of the identified individual microbe to a ruminant lacking in endogenous microbes capable of degrading the remediable compound, such that the ruminant then is capable of degrading the remediable compound.

20. The method of claim 18 where the consortium of anaerobic microbes is obtained from a rumen of a ruminant animal.

21. The method of claim 18 where the remediable compound is a nitroaliphatic compound, a nitroaryl compound, a nitro-heteroaryl compound, or a combination thereof.

22. A method for identifying anaerobic microbes capable of degrading a remediable compound, including a nitro-based explosive, reaction product thereof, and/or metabolite thereof, comprising:
obtaining a consortium of anaerobic microbes;
encouraging the growth of anaerobic microbes within the consortium that are capable of degrading the remediable compound, where encouraging the growth comprises exposing the consortium for a period of time to a medium comprising the remediable compound, wherein the remediable compound is radio-labeled, and wherein the anaerobic microbes incorporate the radio-label from the remediable compound to produce radio-labeled nucleic acids;
after the period of time, extracting nucleic acids from the anaerobic microbes, where the nucleic acids include radio-labeled 16S RNA;
separating the radio-labeled 16S RNA from other nucleic acids;
preparing cDNA from the radio-labeled 16S RNA;
cloning the cDNA into a vector;
amplifying and extracting the cDNA from the vector;
obtaining a DNA sequence of at least a portion of the cDNA;
using the DNA sequence to identify one or more individual microbes capable of degrading the remediable compound; and
administering an effective amount of the identified individual microbe to a ruminant, such that the ruminant then is capable of degrading the remediable compound.
